(19) United States
(12) Patent Application Publication
(10) Pub. No.: US 2026/0201426 A1
(43) Pub. Date: Jul. 16, 2026

(54) EX-SITU BIOMETHANATION METHOD

(71) Applicants: UNIVERSITE CLERMONT AUVERGNE, Clermont-Ferrand (FR); CENTRE NATIONAL DE LA RECHERCHE SCIENTIFIQUE, Paris (FR); CLERMONT AUVERGNE INP, Aubiere (FR)

(72) Inventors: Pierre FONTANILLE, Ceyrat (FR); Christophe VIAL, Clermont-Ferrand (FR); Jean-Sebastien GUEZ, Veyre-Monton (FR); Khaled FADHLAOUI, Beaumont (FR); Pascal DUBESSAY, Aubiere (FR); Alina-Violeta URSU, Clermont-Ferrand (FR); Misagh KERAMATIF SHEY JANI, Clermont-Ferrand (FR)

(21) Appl. No.: 19/127,814

(22) PCT Filed: Nov. 6, 2023
(86) PCT No.: PCT/EP2023/080873

§ 371 Date: May 7, 2025

(30) Foreign Application Priority Data
Nov. 7, 2022 (FR) FR2211561

(51) Int. Cl. C12P 5/02; C12M 1/107; C12M 1/12

(52) U.S. Cl. CPC C12P 5/023; C12M 21/04; C12M 25/14

(57) ABSTRACT

The present invention relates to an ex-situ biomethanation method as well as to the device for implementing the same.

The invention relates to an ex-situ methane production method comprising the steps of placing in contact at least one methanogenic microorganism, a culture substrate and optionally a first culture medium in a gas/liquid bioreactor optionally comprising a second culture medium and producing methane by the reaction of incoming gases with the at least one methanogenic microorganism, characterized in that the culture substrate is optionally immobilized in the gas/liquid bioreactor; and the at least one methanogenic microorganism is fixed to the culture substrate; as well as to the device for carrying out the method.

### TECHNICAL FIELD OF THE INVENTION

The present invention relates to an ex-situ biomethanation method as well as to the device for implementing the same.

The invention relates to an ex-situ methane production method comprising the steps of placing in contact at least one methanogenic microorganism, a culture substrate and optionally a first culture medium in a gas/liquid bioreactor optionally comprising a second culture medium and producing methane by the reaction of incoming gases with the at least one methanogenic microorganism, characterized in that the culture substrate is optionally immobilized in the gas/liquid bioreactor; and the at least one methanogenic microorganism is fixed to the culture substrate; as well as to the device for carrying out the method.

### PRIOR ART

Methanation is a reaction to synthesize methane from dihydrogen and carbon dioxide.

In recent years, methanation has been directly associated with the development of wind and solar energy, which depends on the ability to store electricity produced but not consumed on a massive scale. Power-to-gas conversion is a promising solution for converting this excess electricity into hydrogen by electrolysis of water. However, as the hydrogen sector is still under construction, the conversion of this hydrogen into methane by methanation makes it possible to store this energy on a massive scale using existing gas infrastructure, this is the concept of power-to-methane. The other advantage of methanation is that it captures and stores carbon dioxide during this conversion, thus reducing the environmental impacts linked to carbon dioxide emissions (biogas from mechanization, syngas obtained by pyrolysis or gasification, combustion gas effluent or equivalent). This double advantage that characterises methanation makes it a technology of the future.

There are two competing routes for methanation: the catalytic route, which has been struggling to industrialise for many years, and the biological route, which is more robust (with regard to impurities such as CO, NH₃, H₂S, etc.) and has a smaller environmental footprint, in particular due to reduced energy costs.

However, to date, no in situ or ex-situ biomethanation technology is yet mature or economically viable, and the various dedicated methods need to be further investigated.

The major disadvantage of the biological route lies in the kinetics of methane production, which is slower than by catalytic means. This kinetic lock is partly the result of a limitation by the physicochemical processes of transfer of gaseous dihydrogen material to the liquid phase. Conventional solutions are to increase the pressure and/or intensify the agitation, which can have an antagonistic effect on biological processes due to the sensitivity to mechanical stress of microorganisms, and in particular hydrogenotrophic methanogenic archaea, and can reduce their productivity. There is a need for the industrialization of a biological method with an acceptable energy cost and therefore a reduced economic cost.

### DISCLOSURE OF THE INVENTION

The present invention provides an ex-situ biomethanation method and a device that solve in a surprising way the drawbacks of known biological methods and are an interesting alternative to catalytic methods that are difficult to industrialize. The present invention thus increases the robustness and longevity of the method at lower pressure and temperature compared to the catalytic route, but also increases the methane content of the outgoing gas, and thus the conversion efficiency, productivity and robustness of biological methods by reducing the need for external energy supply compared to known prior art technologies.

A first object of the present invention is an ex-situ methane production method comprising the following steps:
a) placing in contact at least one methanogenic microorganism, a culture substrate and optionally a first culture medium;
b) introducing the mixture obtained in step a) into a gas/liquid bioreactor optionally comprising a second culture medium;
c) contacting incoming gases in the gas/liquid bioreactor obtained in step b);
d) reaction of incoming gases with the at least one methanogenic microorganism;
e) recovery of outgoing gases obtained in step d);
characterized in that
   the culture substrate is optionally immobilized in the gas/liquid bioreactor; and
   at least one methanogenic microorganism is fixed to the culture substrate.

Advantageously, steps a) and b) can occur sequentially or simultaneously.

Advantageously, steps c), d) and e) can occur sequentially and/or simultaneously.

“At least one methanogenic microorganism” is defined herein as a consortium of microorganisms comprising at least one methanogenic strain or one pure methanogenic strain.

Advantageously, the at least one methanogenic microorganism may comprise a microorganism belonging to the phylum Euryarchaeota and may be selected from the classes Methanobacteria, Methanococci, Methanopyri or Methanomicrobia. Preferably, the at least one methanogenic microorganism may comprise at least one strain of Methanothermobacter.

### Reference to Deposited Biological Material

In a particularly advantageous embodiment of the invention, the at least one methanogenic microorganism is the strain of Methanothermobacter marburgensis CLERMONT filed under the Budapest Treaty on Oct. 20, 2022; with the DSMZ (Leibniz Institute Deutsche Sammlung von Mikroorganismen und Zellkulturen GmbH, Inhoffenstraße 7B 38124 Braunschweig GERMANY) under number DSM 34405.

The at least one methanogenic microorganism can be comprised in a preculture liquid. The preculture medium can be the same or different from the first culture medium.

By “culture substrate” it is meant a medium that allows it to be colonised by hydrogenotrophic and/or methanogenic microorganisms. The substrate can be in the form of balls, chips, rollers, gel, foam, pellets, rings, tower or biochips. Preferably, the culture substrate can be spherical or pseudo-spherical.

Advantageously, the culture substrate can be an organic or inorganic substrate, of natural or synthetic origin.

Advantageously, the organic, naturally derived culture substrate can be selected from: alginate, K-carrageenan, chitosan, sawdust, straw, charcoal, plant fibers, corn cob, bagasse, rice, sunflower seed husks, diatomite, mycelium and a mixture thereof.

Advantageously, the organic culture substrate, of synthetic origin can be a polymer. The polymer can be expanded or non-expanded. The polymer can be selected from: polyethylene, polypropylene, polyvinyl chloride, polystyrene, polyurethane, polyacrylonitrile, polyvinyl alcohol, polyamide (PA), and polylactic acid (PLA) and a mixture thereof. Preferably, the organic culture substrate, of synthetic origin is an expanded polymer. Preferably, the culture substrate can be selected from porous cubes made of polyurethane foam impregnated with powdered activated charcoal and/or rigid polyethylene biochips.

Advantageously, the inorganic culture substrate, of natural or synthetic origin, can be selected from: magnetite, volcanic rocks, vermiculite, porous glass, silica-based materials, ceramics, nanoparticles and a mixture thereof. Preferably, the culture substrate can be selected from sepiolite, pozzolan and/or porous glass (e.g. expanded glass that can be marketed under the name Poraver (registered trademark)). More preferably, the culture substrate can be selected from sepiolite, pozzolan and/or porous glass and be spherical or pseudo-spherical in shape.

Advantageously, the culture substrate can be selected from porous cubes made of polyurethane foam impregnated with powdered activated charcoal, rigid polyethylene biochips, sepiolite, pozzolan and/or porous glass.

By “optionally immobilised”, it is herein meant in the case “immobilized” that all the elements of the solid substrate do not move relative to each other or with respect to the reactor body (in the case of a descending liquid flow, an upward liquid flow at low velocity for a solid with a density greater than that of the liquid, or solid substrates whose movement is mechanically blocked, for example by means of a grid). On the contrary, “non-immobilized” means that the solid substrates are suspended in an upward liquid flow, in “fluidized bed” mode, moving relative to each other and to the reactor body, but that the net velocity of all the solid substrates is zero with respect to the reactor body.

By “at least one methanogenic microorganism is fixed to the culture substrate” it is herein meant that the microorganism is not free in its environment (culture medium or otherwise) and that when the at least one methanogenic microorganism fixed to the substrate comes into contact with a culture medium, a system with two distinct phases appears. Generally, from 0.01 to 100% (by weight) of the at least one methanogenic microorganism is fixed to the substrate, preferably from 60 to 100% and more preferably from 80 to 100%.

By “culture medium” it is meant a culture medium in which the at least one microorganism can be maintained to generate a gas mixture, and into which incoming gases will be injected and dissolved, whether or not the medium can produce biomass.

Advantageously, the culture medium can comprise water, nutrients, trace elements, or a mixture thereof. Preferably, the culture medium may comprise sources of nutrients (nitrogen, calcium, sodium, potassium, sulphur, phosphorus, magnesium) and trace elements (iron, zinc, copper, cobalt, nickel, molybdenum, iodine and boron) necessary for the maintenance of microorganisms or for the growth of microorganisms and microbial activity.

Advantageously, the culture medium can be a continuous liquid phase. “Continuous liquid phase” within the meaning of the present invention means a volume of liquid having physical continuity, as opposed to a discontinuous liquid volume consisting of a set of liquid phases without contact with each other such as liquid droplets percolating in a gas phase.

Advantageously, the pH of the culture medium can be in the range from 7 to 9. Preferably, the pH of the culture medium can be 8.

Advantageously, at least one of the steps a) or b) can implement at least one culture medium. The placing in contact of step a) can be carried out between at least one methanogenic microorganism and a culture substrate before the obtained mixture is introduced in step b) into a gas/liquid bioreactor comprising a culture medium. The placing in contact in step a) can be carried out between at least one methanogenic microorganism, a culture substrate and a culture medium before the obtained mixture is introduced in step b) into a gas/liquid bioreactor which does not yet comprise a culture medium. The placing in contact of step a) can be carried out between at least one methanogenic microorganism, a culture substrate and a first culture medium before the obtained mixture is introduced in step b) into a gas/liquid bioreactor comprising a second culture medium. The first and second culture medium can be the same or different.

Advantageously, the method according to the invention can be continuous, semi-continuous or discontinuous (batch operation).

In a first variant, the ex-situ methane production method according to the invention comprises the following steps:
a) placing in contact at least one methanogenic microorganism with a culture substrate;
b) introducing the mixture obtained in step a) into a gas/liquid bioreactor comprising a culture medium;
c) contacting incoming gases in the gas/liquid bioreactor obtained in step b);
d) reaction of incoming gases with the at least one methanogenic microorganism;
e) recovery of outgoing gases obtained in step d);
characterized in that
   the culture substrate is optionally immobilized in the gas/liquid bioreactor; and
   at least one methanogenic microorganism is fixed to the culture substrate.

In a second variant, the -situ methane production method according to the invention comprises the following steps:
a) placing in contact at least one methanogenic microorganism, a culture substrate and a culture medium;
b) introducing the mixture obtained in step a) into a gas/liquid bioreactor;
c) contacting incoming gases in the gas/liquid bioreactor obtained in step b);
d) reaction of incoming gases with the at least one methanogenic microorganism;
e) recovery of outgoing gases obtained in step d);
characterized in that
   the culture medium is optionally immobilized in the gas/liquid bioreactor; and
   at least one methanogenic microorganism is fixed to the culture substrate.

In a third variant, the ex-situ methane production method according to the invention comprises the following steps:
a) placing in contact at least one methanogenic microorganism, a culture substrate and a first culture medium;
b) introducing of the mixture obtained in step a) into a gas/liquid bioreactor comprising a second culture medium;
c) contacting incoming gases in the gas/liquid bioreactor obtained in step b);
d) reaction of incoming gases with the at least one methanogenic microorganism;
e) recovery of outgoing gases obtained in step d);
characterized in that
   the culture medium is optionally immobilized in the gas/liquid bioreactor; and
   at least one methanogenic microorganism is fixed to the culture substrate.

By “gas/liquid bioreactor” it is herein meant a fermenter capable of implementing a biological reaction requiring the dissolution of at least one reagent present in a gaseous state in a culture medium.

Advantageously, the gas/liquid bioreactor can be selected from a pneumatically stirred reactor such as a bubble column with upward or downward liquid circulation or an airlift reactor, a mechanically stirred column with upward or downward liquid circulation, a continuous stirred-tank reactor (CSTR), a flooded fixed bed reactor, a fluidized bed reactor, a sprinkled bed reactor. Preferably, the gas/liquid bioreactor can be selected from a pneumatically stirred reactor, preferably with a downward liquid circulation.

Advantageously, steps a) and b) of the method according to the invention can be carried out at a temperature comprised between 25 and 70° C. Preferably, the temperature of steps a) and b) can be 55° C.

Advantageously, steps a) and b) of the method according to the invention can be carried out under a pressure comprised between 1 and 20 bar (absolute pressure). Preferably, the pressure of steps a) and b) can be comprised between 1 and 3 bar. More preferably, the pressure of steps a) and b) can be 2 bar.

Advantageously, the method according to the invention may also comprise an intermediate step a′), implemented between steps a) and b) allowing the proliferation of the at least one methanogenic microorganism on the substrate before its introduction into the gas/liquid bioreactor. The proliferation step a′) may last less than or equal to 6 months. Preferably, the duration of step a′) can be 30 days.

Steps a) and a′) allow the formation of a methanation catalyst (substrate colonized by at least one methanogenic microorganism). At the end of step a), or optionally a′), the methanation catalyst may or may not be isolated from the first liquid medium before the implementation of step b). Thus, the “mixture obtained in step a)” may represent the mixture comprising the catalyst and the first culture medium, or the catalyst only, when the culture medium is absent from step a), or when it has been isolated therefrom.

Advantageously, the method according to the invention may also comprise an intermediate step b′), implemented between steps b) and c) allowing the proliferation of the at least one methanogenic microorganism on the substrate in the gas/liquid bioreactor before the introduction of incoming gases. The proliferation step b′) may last less than or equal to 3 months. Preferably, the duration of step b) can be 15 days.

Advantageously, the incoming gases of step c) of the method according to the invention can be carbon dioxide (CO₂) and dihydrogen (H₂). The incoming gases are not limited to CO₂ and H₂. They may comprise any other gas, such as methane, carbon monoxide, nitrogen, ammonia and/or hydrogen sulphide. The H₂/CO₂ volume ratio can be comprised in the range from 2:1 to 6:1. Preferably, the H₂/CO₂ volume ratio can be 4:1.

Advantageously, the flow rate of the incoming H₂ of step c), d) or e) of the method according to the invention can be comprised in a range from 0.1 to 10 NL/L_{reactor}/h, which is equivalent to 2.4 to 240 NL/L_{reactor}/day. Preferably, the flow rate of incoming H₂ may be 10 NL/L_{reactor}/h, which is equivalent to 240 NL/L_{reactor}/day.

Advantageously, the flow rate of the incoming CO₂ of step c), d) or e) of the method according to the invention can be comprised in a range from 0.025 to 2.5 NL/L_{reactor}/h, which is equivalent to 0.6 to 60 NL/L_{reactor}/day. Preferably, the flow rate of incoming CO₂ may be 2.5 NL/L_{reactor}/h, which is equivalent to 60 NL/L_{reactor}/day.

Advantageously, the liquid is recirculated in a loop in the reactor, via a recirculation loop in order to homogenize the reaction medium. The recirculation rate of the liquid is comprised in a range from 0.1 to 1 L/L_{reactor}/min.

Advantageously, the reaction temperature of step d) of the method according to the invention can be comprised in a range from 25 to 70° C. Preferably, the temperature can be 55° C.

Advantageously, the pressure inside the bioreactor during steps c), d) and e) of the method according to the invention can be comprised in a range from 1 to 20 bar. Preferably, the pressure of step c), d) and e) can be comprised between 2 and 6 bar or between 1 and 3 bar. More preferably, the pressure of step c), d) and e) can be 2 bar.

Advantageously, the outgoing gases may comprise methane (CH₄). The outgoing gases may also comprise water vapor.

Advantageously, the volume ratio of methane (CH₄) to other outgoing gases (CO₂ and H₂) can be comprised in the range of 2:3 to 9.9:10.

Advantageously, the flow rate of the outgoing gases can be comprised in a range from 0.025 to 2.5 NL/L_{reactor}/h, which is equivalent to 0.6 to 60 NL/L_{reactor}/day. Preferably, the flow rate of the outgoing gases can be 2.5 NL/L_{reactor}/h, which is equivalent to 60 NL/L_{reactor}/day.

Advantageously, the method according to the invention may also comprise a step d′) of replenishment of the culture medium with nutrients. This step enables to compensate for the decrease in the concentration of nutrients in the culture medium, which are consumed by the microorganisms during methane production in step d) of the method.

A second object of the present invention is a device 1 for the ex-situ methane production comprising:
a gas/liquid bioreactor 11 comprising a vessel 12, a gas inlet 13, a gas outlet 14, a liquid inlet 15 and a liquid outlet 16, at least one methanogenic microorganism 121 fixed on a culture substrate 122, optionally immobilized, and a culture medium 123;
a gas injection means 17 for injecting incoming gases into said continuous liquid phase 123 contained in the gas/liquid bioreactor 11;
a liquid injection means 18 for injecting said continuous liquid phase 123 into the gas/liquid bioreactor 11;
a recovery means 19 to recover outgoing gases from the gas/liquid bioreactor 11.

The definitions described above also apply to the mentioned device, where applicable.

Advantageously, the volume of the bioreactor vessel 12 can be comprised in a range from 0.01 to 150 m³. Preferably, the volume of vessel 12 of bioreactor 11 can be less than 50 m³ for an agricultural facility and more than 50 m³ for an industrial facility. More preferably, the volume of vessel 12 of bioreactor 11 can be 100 m³ for an industrial facility. The height/diameter ratio of vessel 12 can be comprised between 10:1 and 10:3.

By “gas injection means” it is meant any device for injecting incoming gases into the continuous liquid phase contained in a gas/liquid bioreactor.

Advantageously, gas injection means 17 can be selected from fine bubble diffusers such as a porous bottom-of-column diffuser, a pierced tube, a porous membrane made of polymers or ceramic material, a flap bubbler, or from bubble-free membrane contactors such as hollow fiber membranes, or from water ejectors or static mixers. Preferably, the gas injection means 17 can be a porous bottom-of-column diffuser. The gas/liquid bioreactor 11 can be configured to be continuously supplied with incoming gases, especially during steps d) and e). The gas injection means 17 is connected to the gas inlet 13 of bioreactor 11.

Herein, “connected” means a direct or indirect connection between two elements of the device.

By “liquid injection means” it is meant any device for injecting a continuous liquid phase into a gas/liquid bioreactor. The liquid injection means 18 is connected to the liquid inlet 15 of bioreactor 11.

Advantageously, the device according to the invention may comprise a liquid recirculation loop 24 connected to the liquid outlet 16 and to the liquid inlet 15. The recirculation loop can be connected to the purging means 20. The liquid recirculation loop can comprise a pump 23 and the liquid injection means 18. The pump can be a peristaltic pump.

Advantageously, the liquid injection means 18 is selected from multi-orifice static dispersion systems (in the shape of a crown or comb for example). Preferably, the liquid injection means 18 can be a comb-shaped multi-orifice.

Advantageously, device 1 can also comprise a purging means 20 to purge the said continuous liquid phase comprised in the gas/liquid bioreactor 11. Purging means 20 is connected to the liquid outlet 16 of bioreactor 11.

The gas/liquid bioreactor 11 can be configured so that nutrient input and/or purge of the culture medium is carried out continuously or discontinuously.

By “recovery means” it is meant any device for recovering outgoing gases from a gas/liquid bioreactor.

Advantageously, the recovery means 19 can be selected from a simple gas outlet, a gas outlet with a condenser (in particular to eliminate the residual water vapour contained in the outgoing gases), an outlet associated with a recirculation system for the outgoing gases. Preferably, the recovery means 19 can be an outlet associated with a recirculation system for the outgoing gases. The gas/liquid bioreactor 11 can be configured so that the recovery of outgoing gases is carried out on a continuous basis. The recovery means 19 is connected to the gas outlet 14 of bioreactor 11. The water vapor condensates can either be reinjected into bioreactor 11 or extracted out of device 1. As the methanation reaction produces water, there is an advantage of being able to modulate the quantity of water in device 1.

Advantageously, device 1 can comprise a gas outlet 14 equipped with a condenser.

Advantageously, device 1 can comprise a gas outlet 14 equipped with a meter.

Advantageously, device 1 can comprise a gas loop connected to analyzers, preferably chromatograph-type analyzers.

Advantageously, device 1 can comprise a gas recirculation loop from the upper part of bioreactor 11 to the lower part.

Advantageously, device 1 can comprise a mixer of the recirculated gases with the incoming gases.

Advantageously, device 1 can comprise a set of probes for verifying the concentration of an incoming gas, the redox potential or the pH in culture medium 123 (directly in bioreactor 11). The pH probe is an advantageous way to measure temperature.

Advantageously, device 1 can comprise gas bottles allowing the supply of incoming gases. The bottles can be connected to gas injection means 17. The gas injection means 17 may also comprise a mixer, so as to control the CO₂/H₂ ratio of the incoming gases. The said mixer may be the same as or different from the mixer of the recirculated gases with the incoming gases.

By “bottles” it is meant a gas storage means; the said storage means may be substituted by any other compatible storage means.

Advantageously, device 1 can comprise a set of flow meters, preferably mass flow meters. These mass flow meters allow the adjustment of the inlet flow rates of incoming gases.

Advantageously, device 1 can comprise a gas meter.

Advantageously, device 1 can comprise a sampling means. This sampling means makes it possible to take samples of liquid for the analysis of compounds and to monitor the progress of the method, for example.

Advantageously, Device 1 can comprise a means of sampling the composition of the outgoing gas mixture for the analysis of the compounds.

Advantageously, device 1 can comprise a set of valves.

Advantageously, device 1 can comprise a pH regulation system.

Advantageously, device 1 can comprise a sub-device housed inside the vessel so that it can be compartmentalized by means of a grid. Preferably, the sub-device is present in the case where several culture substrates of a different nature are used.

The invention also relates to a kit, said kit once assembled allowing to obtain device 1 according to the invention, comprising:
a substrate 122;
at least one methanogenic microorganism 121;
optionally a gas/liquid bioreactor 11;
optionally a gas injection means 17;
optionally a liquid injection means 18;
optionally a recovery means 19.

The invention also relates to a methanation catalyst comprising a substrate 122 obtained in step a) of the method according to the invention. The catalyst comprises at least one methanogenic microorganism 121 fixed to a substrate 122.

Another object of the present invention relates to the use of the strain of Methanothermobacter marburgensis CLERMONT DSM 34405 for the production of methane.

Another object of the present invention relates to the use of the strain of Methanothermobacter marburgensis CLERMONT DSM 34405 for the implementation of a biomethanation method.

Other advantages will appear in the light of the following examples, given for illustrative purposes and not exhaustively, with reference to the annexed figures.

### BRIEF DESCRIPTION OF THE FIGURES

FIG. 1 represents a device 1 for the production of methane according to the present invention comprising:
a gas/liquid bioreactor 11 comprising a vessel 12, a gas inlet 13, a gas outlet 14, a liquid inlet 15 and a liquid outlet 16, at least one methanogenic microorganism 121 fixed on a culture substrate 122, a culture medium 123, a gas injection means 17, a gas recovery means 19 and a liquid recirculation loop 24, connected to the liquid inlet 15 and to the liquid outlet 16, comprising a pump 23 and a liquid injection means 18 for injecting said continuous liquid phase 123 into the gas/liquid bioreactor 11
two incoming gas sources connected by two valves 21 to gas injection means 17;
a chromatograph 22 connected by a valve 21 to the recovery means 19;
a purging means 20 connected to the recirculation loop 24 and comprising a valve 21.

FIG. 2 represents the evolution over time of the growth of bacteria and archaea in a stirred bioreactor, in the absence (▪ without substrate) or in the presence of polyethylene culture substrates at a low (● FR_low) or high (▴ FR_high) filling rate, in the presence of the modified basal BA medium at 100 RPM and at 55° C., with H₂ and CO₂ supplementation.

FIG. 3 represents the evolution of the total number of archaea and bacteria in the absence (without substrate, left) or in the presence of culture substrates at a low (FR_low, center) or high (FR_high, right) filling rate of polyethylene culture substrates. Culture in a stirred bioreactor is carried out in the presence of the modified basal BA medium, at 100 RPM and 55° C., with H₂ and CO₂ supplementation.

FIG. 4 is an image of a methanation catalyst in polyethylene biochips taken with SEM at 0 days of growth of Methanothermobacter marburgensis Clermont DSM 34405, in modified BA medium, at 55° C. with H₂ and CO₂ supplementation.

FIG. 5 is an image of a methanation catalyst in polyethylene biochips taken with SEM at 14 days of growth of Methanothermobacter marburgensis Clermont DSM 34405, in modified BA medium, at 55° C. with H₂ and CO₂ supplementation.

FIG. 6 is an image of a methanation catalyst in polyethylene biochips taken with SEM at 34 days of growth of Methanothermobacter marburgensis Clermont DSM 34405, in modified BA medium, at 55° C. with H₂ and CO₂ supplementation.

### EXAMPLES

Other advantages, aims and special features of the present invention will be apparent from the following examples, which are made for explanatory and not limited purposes.

In the following examples, the different parameters were measured using the following detailed techniques:

### Measurement of the Tolerance of the at Least One Methanogenic Microorganism to Exposure to Oxygen (O₂)

Several exposures to the open air lasting 1 min, 10 min, 30 min, 60 min and 180 min are carried out in the enclosure of a laminar flow hood (PSM II Cytosafe, Faster).

For each duration of exposure, three vials containing 30 mL of culture medium containing the at least one methanogenic microorganism are inoculated with 3 mL of a culture medium containing at least one methanogenic microorganism in the exponential growth phase. To restore anaerobiosis, the O₂ is removed by purging the vials with the H₂/CO₂ mixture, the pressure is then adjusted to 1.5 bar. The cultures are incubated at 55° C. for 10 days. The ability to grow again after exposure to O₂ is monitored by CH₄ dosage and by spectrophotometry (Cell Density Meter, Fisherbrand).

### Measurement of the Gas Phase Composition

The composition of the gas phase is continuously analyzed by gas chromatography (Agilent analyzer, 3000A® Technologies) equipped with two separation modules and a thermal conductivity detector (TCD). Module A equipped with a 5 Å molecular sieve separates hydrogen, nitrogen, oxygen and methane under an argon stream. Module B equipped with a PoraPlot U (Agilent) column allows the separation of carbon dioxide and hydrogen sulphide, under hydrogen flow. A hydrophobic filter is placed at the inlet of the analyzer to protect it from humidity. The bioreactor's gas analyses are carried out every hour.

### Measurement of the Content of Metabolites and Organic Acids

The content of metabolites and organic acids is analyzed by liquid chromatography (HPLC Agilent Technologies, 1260 Infinity). Separation is provided by two ion exclusion columns (Rezex ROA 300x7, 8 nm, Phenomenex, USA) mounted in series and heated to 50° C. The detector used to identify the different compounds is a refractometer (HP 1100 series). The liquid phase consists of a solution of sulphuric acid (2 mM) circulating at a rate of 0.7 mL/min. Before injection, the samples are deproteinized. A volume of 1 mL of culture sample is mixed with 125 μL of Ba(OH)₂ 8H₂O (0.3 M) and 125 μL of ZnSO 7H₂O (5% w/v) and centrifuged (5 min; 10,000 g). The supernatant is then filtered to 0.22 μm and transferred to an HPLC vial stored at 4° C. before injection.

### Measurement of the Growth of Microorganisms

The evolution of the growth of microorganisms is monitored by spectrophotometry (Uvisco, V-1800) at 600 nm.

### Microscopic Observation

A volume of 0.5 ml of a culture on BA (Basal anaerobic) medium described by Bu et al., (reference 1) and then modified, was collected and fixed with a formaldehyde solution (2%, final concentration). The cells were then seasoned by centrifugation at 18000 g (20 min, 14° C.) on 400-mesh copper grids coated with carbon (Formvar, Pelanne Instruments, France) and then contrasted by immersion in 20 μL of 2% uranyl acetate. After rinsing with distilled water and drying on absorbent paper, the observations were made with a JEOL 2100 plus transmission electron microscope (TEM) (Akishikma, Tokyo, Japan, UCA Partner CYSTEM platform) equipped with a Gatan CMOS RIO 9 camera (Gatan Ametek, Pleasanton, USA) at an acceleration voltage of 80 kV. Samples from the culture substrates were fixed with a solution of 2.5% glutaraldehyde and 0.15% ruthenium red, in 0.2M sodium cacodylate buffer, pH 7.4 overnight at 4° C. After rinsing in the same buffer (3×10 min), they are post-fixed for 1 hour at room temperature with 1% osmic acid in 0.2M sodium cacodylate buffer, pH 7.4. Then the samples are rinsed with distilled water for 20 minutes and dehydrated by ethanol baths of increasing degree (25° to 100°), 10 minutes/bath. The last dehydration step is carried out with a mixture of 100° ethanol V/V and hexamethyldisilazane for 10 minutes and then pure hexamethyldisilazane (evaporation overnight under a fume cupboard). The samples are then placed on a metal pad using double-sided carbon tape. Chromium metallization (5 nm) is performed with the Quorum Q150 TES Plus metallizer. Observations are made using the Hitachi Regulus 8230 scanning electron microscope at an acceleration voltage of 1 kV and with the secondary electron detector.

### Example 1: Evaluation of the Tolerance of the Strain Methanothermobacter marburgensis Clermont DSM 34405 to Exposure to Oxygen (O₂)

The simplification of a consortium from a methanisation unit (Ennezat, 63) by selecting hydrogenotrophic microorganisms has made it possible to isolate a new methanogenic strain that overproduces methane.

To isolate the archaeal strain with the highest content in the bioreactor, a 50 mL sample from the consortium from the digestate of a methanization unit (Ennezat, 63) is taken under sterile and anaerobic conditions. In some cases, the sample is cultured under selective conditions of methanogenic hydrogenotrophic strains, in the presence of an H₂/CO₂ mixture in a 4:1 ratio at a temperature of 55° C. for a period of 6 weeks. After an incubation of 72 hours at 55° C. (bioreactor temperature), a transplant is carried out in a modified BA medium (10% inoculation: 0.5 mL of inoculum in 5 mL of culture medium). Serial dilutions (from 10-1 to 10-10) are carried out in accordance with the physicochemical conditions of the bioreactor (55° C., 1.5 bar and pH=7.5). The tube of the last positive dilution (turbidity and CH₄ production) is used as an inoculum to inoculate the next series. Growth is monitored by spectrophotometry (Cell Density Meter, Fisherbrand) and gas assay by microchromatography (Agilent analyzer, 3000A® Technologies) equipped with two separation modules and a thermal conductivity detector (TCD). Module A with a 5 A molecular sieve separates hydrogen, nitrogen, oxygen and methane under an argon stream. Module B equipped with a PoraPlot U (Agilent) column allows the separation of carbon dioxide and hydrogen sulfide, under hydrogen flow, in the gas phase. Isolation on a solid medium is then carried out by adopting the Roll-tube method (Hungate, 1969). To do this, 0.1 g of agar-agar (Bacto™ Agar) is added to 5 mL of modified BA medium (Hungate tube), under N₂ flow, and then autoclaved. The agar medium is then liquefied in a water bath at 100° C., then placed at 45° C. in order to slow down its gelling. After inoculation by the last series of positive dilution in culture medium, the agar medium is uniformly arranged in a thin layer on the inner side of the tube by the rotary movement of the Spinner tube and by using an ice cube on the outer surface to solidify the medium. The tubes are then incubated at 55° C. for 168 hours in a vertical position to avoid contamination of the colonies due to condensation. Tubes showing the presence of a parent microorganism entity (colony), visible to the naked eye, comprising a large number of identical microorganisms, and CH₄ production, are opened in a controlled atmosphere (atmospheric pressure) anaerobic chamber to collect colonies using a sterile Pasteur pipette. Colonies are resuspended in 5 mL of culture medium. The purity of the liquid cultures is confirmed by performing several successive serial dilutions (10-1 to 10-5) and optical microscope checks (Labophot, Nikon). Analyses can be carried out on the isolated strain.

Table 1: Influence of oxygen exposure on the growth and metabolic activity of Methanothermobacter marburgensis isolate CLERMONT DSM 34405

TABLE 1

| — | Day 0 | | | Day 5 | | | Day 10 | | |
|---|---|---|---|---|---|---|---|---|---|
| — | H₂ (% | CO₂ (% | CH₄ (% | H₂ (% | CO₂ (% | CH₄ (% | H₂ (% | CO₂ (% | CH₄ (% |
| | volume) | volume) | volume) | volume) | volume) | volume) | volume) | volume) | volume) |
| 0 | 79.12 | 20.14 | 0 | 0.12 | 1.65 | 53.64 | 0 | 1.2 | 53.47 |
| min | +/− | +/− | | +/− | +/− | +/− | +/− | +/− | +/− |
| | 2.14 | 1.34 | | 2.64 | 2.45 | 5.14 | 0.14 | 0.87 | 3.45 |
| 1 | 80.14 | 19.14 | 0 | 0.34 | 0.57 | 55.64 | 0.08 | 0.15 | 56.20 |
| min | +/− | +/− | | +/− | +/− | +/− | +/− | +/− | +/− |
| | 1.84 | 0.39 | | 0.27 | 1.23 | 3.14 | 0.4 | 1.45 | 5.34 |
| 10 | 79.64 | 21.14 | 0 | 0.47 | 2.64 | 60.45 | 0.12 | 1.34 | 61.24 |
| min | +/− | +/− | | +/− | +/− | +/− | +/− | +/− | +/− |
| | 2.54 | 2.64 | | 3.12 | 0.14 | 2.47 | 0.24 | 1.47 | 7.94 |
| 30 | 81.34 | 19.54 | 0 | 1.25 | 0.14 | 51.85 | 0.34 | 0.18 | 52.31 |
| min | +/− | +/− | | +/− | +/− | +/− | +/− | +/− | +/− |
| | 0.64 | 1.45 | | 3.45 | 1.45 | 4.37 | 0.51 | 2.45 | 2.47 |
| 60 | 77.18 | 20.34 | 0 | 0.45 | 3.34 | 50.14 | 0.04 | 0.16 | 51.34 |
| min | +/− | +/− | | +/− | +/− | +/− | +/− | +/− | +/− |
| | 2.45 | 0.47 | | 1.41 | 1.45 | 6.37 | 0.12 | 1.22 | 3.08 |
| 180 | 80.35 | 19.37 | 0 | 11.57 | 5.61 | 41.18 | 0.51 | 1.2 | 53.64 |
| min | +/− | +/− | | +/− | +/− | +/− | +/− | +/− | +/− |
| | 1.86 | 1.84 | | 4.15 | 1.89 | 3.14 | 0.41 | 0.46 | 2.54 |

It appears that exposure to oxygen according to the method described above did not have a significant effect on the growth and hydrogenotrophic and methanogenic activities of the Methanothermobacter marburgensis CLERMONT DSM 34405 strain between 0 and 60 min of exposure. Slight growth retardation is observed after 180 min of exposure to oxygen (Table 1). As the methanogenic performance of the Methanothermobacter marburgensis CLERMONT DSM 34405 strain is not or only slightly affected by exposure to oxygen and therefore air, the different modalities of passage from step a) to step b) can be carried out under non-anaerobic conditions without impact on steps c), d) and e).

### Example 2: Evaluation of the Methanation Catalyst from Step a) of the Method According to the Invention

The ex-situ methane production method implemented comprises the following step:
a) placing in contact of a consortium, a polyethylene biochip culture substrate and a culture medium.

The consortium from a methanisation unit (Ennezat, 63) is taken from the anaerobic digestion tank. The liquid or digestate sample is sieved (5 mm) to remove solids>5 mm and frozen.

The culture medium used is derived from the BA medium described by Bu et al., 2018 and its composition is described in Table 2. The culture medium is adjusted to pH 7.5 with a 3 M NaOH solution.

Table 2: List of compounds in modified BA medium.

TABLE 2

| Compound | Concentration (g/L) |
|---|---|
| NH₄Cl | 1.00 |
| NaCl | 0.10 |
| MgCl₂•6H₂O | 0.10 |
| CaCl₂•2H₂O | 0.05 |
| Na₂S 9H₂O | 1.0 |
| K₂HPO₄•3H₂O | 0.40 |
| Resazurin | 0.0005 |
| L-Cysteine | 0.5 |
| FeCl₂•4H₂O | 0.002000 |
| H₃BO³ | 0.000050 |
| ZnCl₂ | 0.000050 |
| CuCl₂•2H₂O | 0.000038 |
| MnCl₂ 4H₂O | 0.000050 |
| (NH₄)₆Mo₇O₂₄•4H₂O | 0.000050 |
| AlCl₃ | 0.000050 |
| CoCl₂•6H₂O | 0.000050 |
| NiCl₂•6H₂O | 0.000092 |
| EDTA | 0.000500 |
| Na₂SeO₃•5H₂O | 0.000100 |
| Biotin | 0.00000200 |
| Folic acid | 0.00000200 |
| Pyridoxal HCl | 0.00001000 |
| Thiamine HCl | 0.00000500 |
| Riboflavin | 0.00000500 |
| Nicotinic acid | 0.00000500 |
| Ca-D-pantothenate | 0.00000500 |
| Cyanocobalamin | 0.00000010 |
| P-aminobenzoic acid | 0.00000500 |
| Thioctic acid | 0.00000500 |

The colonization kinetics of polyethylene biochip substrates by Methanothermobacter marburgensis CLERMONT DSM 34405 is monitored by SEM imaging (FEG SEM, Hitachi Regulus 8230). The process begins when the microbes associated with the surface switch from a reversible attachment mode (which can be unhooked without compromising integrity) to an irreversible attachment mode, followed by the aggregation of the cells and their subsequent proliferation. The cells of the biofilm are enclosed in a matrix of polymers of the EPS (extracellular polymeric substances) type. SEM observation makes it possible to visualize the EPS grains, the matrix, the cells as well as wireframe structures called fibraeum.

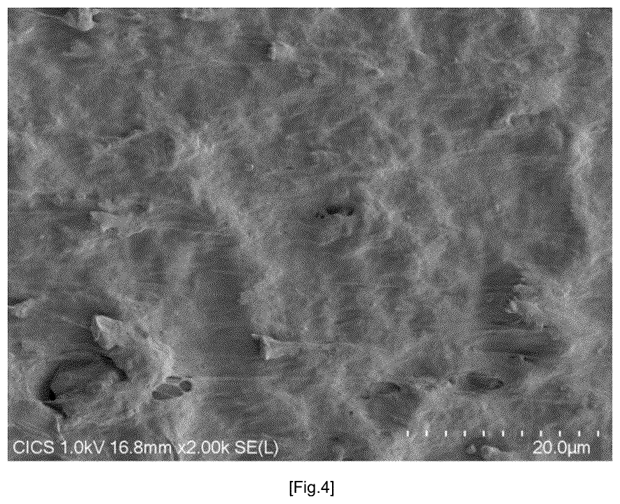

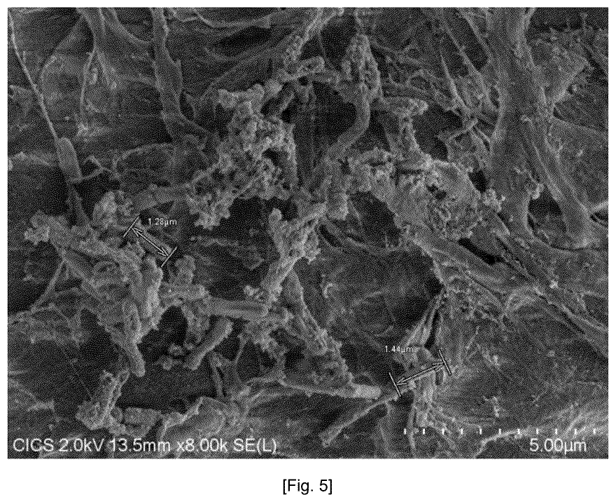

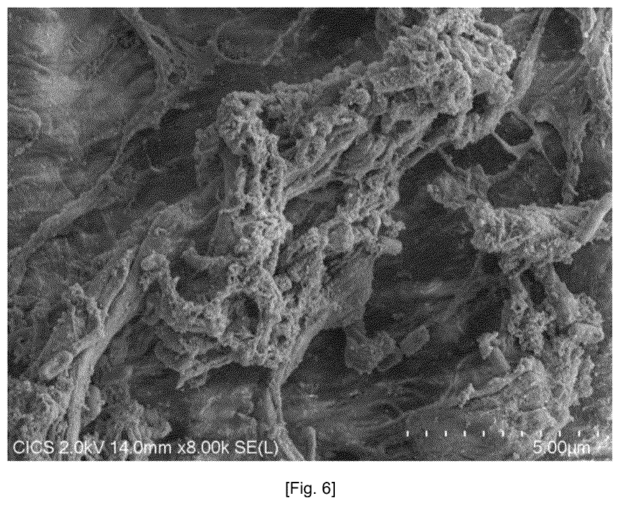

FIGS. 4, 5 and 6 illustrate that the strain Methanothermobacter marburgensis CLERMONT DSM 34405 develops over time when placed in the presence of a polyethylene biochip culture substrate.

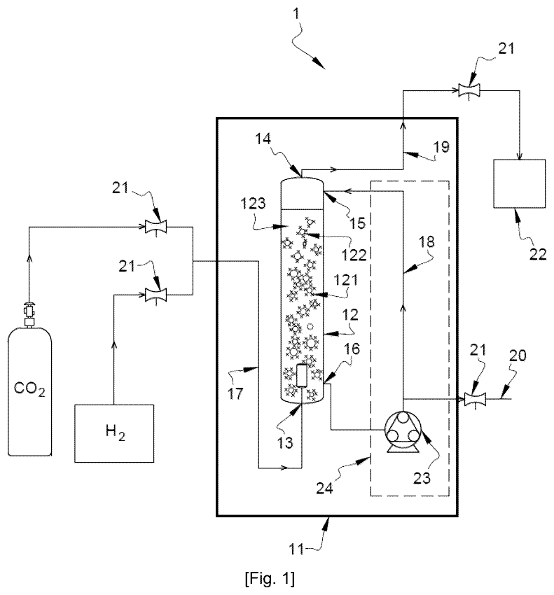

In addition, it appears that the strain Methanothermobacter marburgensis CLERMONT DSM 34405 develops more efficiently when it is placed in the presence of a culture substrate according to the invention in the presence of dihydrogen and carbon dioxide. Indeed, the optical density of the samples overtime is lower in the case where a culture substrate is present (FIG. 2).

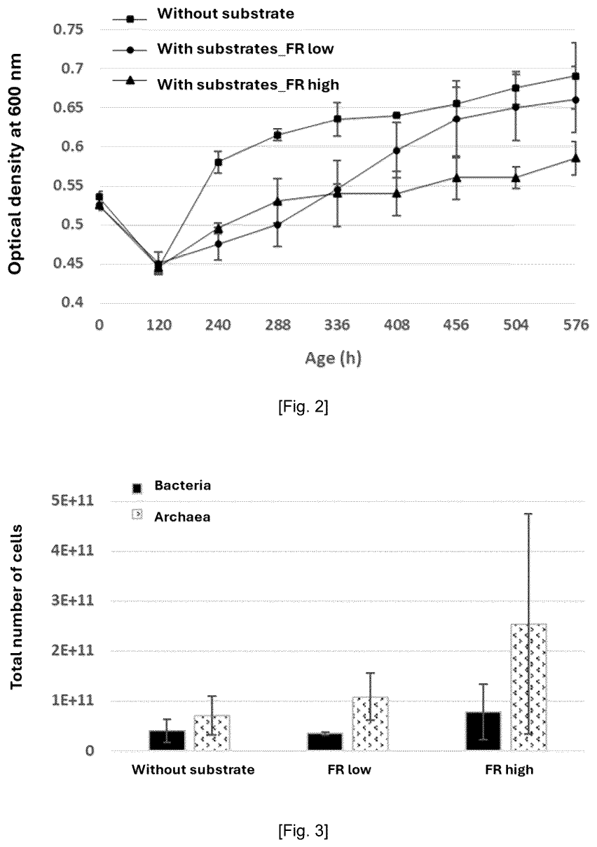

In addition, an analysis of the growth of microorganisms by spectrophotometry (UVisco, V-1800) at 600 nm indicates that the number of microorganisms after 24 days of colonization is three times higher in the presence of a polyethylene biochip culture substrate (FIG. 3).

These results lead to the conclusion that the method according to the invention makes it possible to obtain a better yield in methanation. This is because there is better growth of microorganisms when they are attached to a culture substrate.

### Example 3: Device for Carrying Out the Method According to the Invention

The ex-situ methane production method implemented according to the invention also comprises the following steps:
b) introducing the mixture obtained in step a) (example 2) into a gas/liquid bioreactor;
c) contacting incoming gases in the gas/liquid bioreactor obtained in step b);
d) reaction of incoming gases with the at least one methanogenic microorganism;
e) recovery of outgoing gases obtained in step d);
characterized in that
   the culture substrate is not immobilized in the gas/liquid bioreactor; and
   at least one methanogenic microorganism is attached to the culture substrate.

In the present example, the device as described in FIG. 1 enables the implementation of the method according to the invention.

The bioreactor in stage b) was custom constructed from a section of a 2 mm thick 316 stainless steel cylinder. It has an internal diameter of 54 mm and a height of 1400 mm. The height-to-diameter ratio of the gas/liquid bioreactor of bubble column-type is 25.9. The volume of the vessel is 3.5 L. A glass or stainless steel sintered-type gas distributor of known porosity is coupled to the reactor using a GL18 tri-ring fitting. The gas outlet is through an opening at the top of the column, coupled to the gas circuit by a ¼′G thread. The gas outlet of the reactor is connected to a condenser in which water circulates at 4° C. (Lauda Eco RE1225 silver). A high-precision volume flow meter (Bioprocess control/Microflow 1100-3100) measures the flow of gas leaving the condenser. The gas is then analyzed by gas chromatography (Agilent analyzer, 3000A® Technologies). A culture medium recirculation system based on the use of a peristaltic pump (AB pump, Type PSF2) allows liquid circulation against the rising gas bubbles, at an hourly volumetric speed of 10 L/L_{reactor}/h, which is equivalent to 0.166 L/L_{reactor}/min. The liquid recirculation loop comprises an electrode for measuring oxidation-reduction potential, pH and temperature (Mettler Toledo INPRO4260i/SG/120 52005381). The pH is regulated to 7.5 if necessary by the addition of acidic or basic titrants. The temperature of 55° C. is controlled using a thermostatic bath (Lauda Eco RE1225 silver). The recirculation system is equipped with a septum for sampling, adding stock solutions (culture medium constituents, Na₂S 9H₂O) or withdrawing culture medium volume.

The incoming gases in step c) are dihydrogen and carbon dioxide. They are injected into the bioreactor at a total gas flow rate gradually increasing from 3.61 to 18.21 NL/L_{reactor}/day for CO₂ and gradually increasing from 14.37 to 72.85 NL/L/L_{reactor}/day for H₂ through mass flow meters (Brooks instrument, SLA5800), as shown in Table 3 below. The H₂/CO₂ ratio is adjusted, if necessary, to a value of 4:1±0.1. The pressure of the system is 1 bar. The gas mixture is continuously distributed by a diffuser, against the flow of the aqueous phase.

The outgoing gases of step e) are mainly made up of methane and water vapour (the water vapour being recondensed). They are recovered from the bioreactor at a total outflow rate of outgoing gases gradually increasing from 5.52 to 18.67 NL/L/day as shown in Table 3 below.

Table 3: Evolution of the specific incoming flow rate of CO₂ and H₂ and the specific production of CH₄ (expressed in NL/L_{reactor}/day) as a function of the age of the culture (expressed in days).

TABLE 3

| | H₂ incoming | CO₂ incoming | CH₄ outcoming | H₂ | CO₂ |
|---|---|---|---|---|---|
| | flow rate | flow rate | flow rate | Conver- | Conver- |
| Age | (NL/L_{reactor}/ | (NL/L_{reactor}/ | (NL/L_{reactor}/ | sion | sion |
| (day) | day) | day) | day) | (%) | (%) |
| 1 | 14.37 | 3.61 | 1.79 | 86.19 | 63.76 |
| 2 | 14.37 | 3.61 | 1.72 | 85.36 | 60.96 |
| 3 | 14.37 | 3.61 | 2.12 | 98.32 | 85.17 |
| 4 | 28.99 | 7.27 | 4.72 | 99.51 | 90.67 |
| 5 | 38.74 | 9.70 | 6.73 | 99.37 | 94.00 |
| 6 | 65.54 | 16.40 | 12.05 | 98.15 | 95.98 |
| 7 | 72.85 | 18.21 | 13.44 | 97.59 | 97.16 |
| 8 | 72.85 | 18.21 | 13.05 | 97.63 | 96.12 |
| 10 | 72.85 | 18.21 | 13.77 | 97.96 | 95.96 |
| 13 | 72.85 | 18.21 | 14.01 | 95.64 | 95.18 |

In view of the results of Example 2, the method according to the invention implemented by the device in FIG. 1 allows the production of methane yields superior to conventional processes. This is because the increase in pressure in the system increases the solubility of hydrogen in the culture medium. The presence of substrates colonized by at least one methanogenic microorganism increases the amount of catalyst per unit reactor volume. The cultivated at least one methanogenic microorganism is selected for its ability to produce methane. The presence of substrates colonized by at least one methanogenic microorganism increases the residence time of the gas in the culture medium and promotes the solubility of hydrogen in the liquid. It increases the performance of the system by a factor of 3.6.

### REFERENCES

Reference 1: Bu, F.; Dong, N.; Kumar Khanal, S.; Xie, L.; Zhou, Q. Effects of CO on Hydrogenotrophic Methanogenesis under Thermophilic and Extreme-Thermophilic Conditions: Microbial Community and Biomethanation Pathways. Bioresource Technology 2018, 266, 364-373, doi:10.1016/j.biortech.2018.03.092.

1. A method for the ex-situ methane production comprising the following steps:
a) placing in contact at least one methanogenic microorganism, a culture substrate and optionally a first culture medium and;
b) introducing the mixture obtained in step a) into a gas/liquid bioreactor optionally comprising a second culture medium;
c) contacting incoming gases in the gas/liquid bioreactor obtained in step b);
d) reaction of incoming gases with the at least one methanogenic microorganism;
e) recovery of outgoing gases obtained in step d);
characterized in that
the culture substrate is optionally immobilized in the gas/liquid bioreactor; and
at least one methanogenic microorganism is fixed to the culture substrate.

2. The method according to claim 1, wherein the incoming gases are carbon dioxide, CO₂, and dihydrogen, H₂.

3. The method according to claim 1, wherein the at least one methanogenic microorganism is a consortium or a pure methanogenic strain.

4. The method according to claim 1, wherein the at least one methanogenic microorganism comprises a microorganism belonging to the euryarcheotes phylum and selected from the classes Methanobacteria, Methanococci, Methanopyri or Methanomicrobia.

5. The method according to claim 1, wherein the at least one methanogenic microorganism comprises at least one strain of Methanothermobacter.

6. The method according to claim 1, wherein the gas/liquid bioreactor (11) is selected from a pneumatically stirred reactor with a downward liquid circulation.

7. The method according to claim 1, wherein the culture substrate (122) comprises porous cubes of polyurethane foam impregnated with powdered activated charcoal, rigid polyethylene biochips, sepiolite, pozzolan and/or porous glass.

8. Device (1) for the ex-situ methane production comprising:
a gas/liquid bioreactor (11) comprising a vessel (12), a gas inlet (13), a gas outlet (14), a liquid inlet (15) and a liquid outlet (16), at least one methanogenic microorganism (121) fixed on a culture substrate (122), optionally immobilized, and a culture medium (123);
a gas injection means (17) for injecting incoming gases into said continuous liquid phase (123) contained in the gas/liquid bioreactor (11);
a liquid injection means (18) for injecting said continuous liquid phase (123) into the gas/liquid bioreactor (11);
a recovery means (19) for recovering outgoing gases from the gas/liquid bioreactor (11).

9. The device according to claim 8, wherein the gas/liquid bioreactor (11) is selected from a bubble column, a mechanically stirred column, a continuous stirred-tank reactor (CSTR), an airlift reactor, a flooded fixed bed reactor, a sprinkled bed reactor, a pneumatically stirred reactor, with a downward liquid circulation.

10. The device according to claim 8, wherein the gas/liquid bioreactor (11) is selected from a pneumatically stirred reactor and with a downward liquid circulation.

11. The device according to claim 8, wherein the culture substrate (122) is organic or inorganic, of natural or synthetic origin.

12. The device according to claim 8, wherein the culture substrate (122) comprises porous cubes of polyurethane foam impregnated with powdered activated charcoal, rigid polyethylene biochips, sepiolite, pozzolan and/or porous glass.

13. The device according to claim 8, wherein the at least one methanogenic microorganism (121) comprises at least one strain of Methanothermobacter.

14. (canceled)

15. (canceled)

16. (canceled)

17. Methanation catalyst comprising at least one methanogenic microorganism (121) fixed on a substrate (122), wherein the methanogenic microorganism is a strain of Methanothermobacter marburgensis CLERMONT DSM 34405.

18. The method according to claim 2, wherein the H₂/CO₂ volume ratio is comprised between 2:1 and 6:1.

19. The method according to claim 2, wherein the H₂/CO₂ volume ratio is comprised between 3:1 and 5:1.

20. The method according to claim 2, wherein in the H₂/CO₂ volume ratio is 4:1.

21. The method according to claim 1, wherein the at least one methanogenic microorganism comprises at least one strain Methanothermobacter marburgensis CLERMONT DSM 34405.

22. The device according to claim 8, wherein the culture substrate (122) comprises porous cubes of polyurethane foam impregnated with powdered activated charcoal and/or rigid polyethylene biochips.

23. The device according to claim 8, wherein the at least one methanogenic microorganism (121) comprises at least one strain of Methanothermobacter marburgensis CLERMONT DSM 34405.
